(19) United States
(12) Patent Application Publication
(10) Pub. No.: US 2011/0172579 A1
(43) Pub. Date: Jul. 14, 2011

(54) INFLATION TYPE CERVICAL VERTEBRAE REHABILITATION DEVICE AND METHOD FOR USING THE SAME

(75) Inventors: CHING-HUA CHIU, TAICHUNG CITY (TW); LI-WEI CHOU, TAICHUNG CITY (TW)

(73) Assignee: CHINA MEDICAL UNIVERSITY, TAICHUNG CITY (TW)

(21) Appl. No.: 12/977,619

(22) Filed: Dec. 23, 2010

(30) Foreign Application Priority Data
Jan. 8, 2010 (TW) 99100339

(51) Int. Cl. A61F 5/00

(52) U.S. Cl. 602/36

(57) ABSTRACT

An inflation type cervical vertebrae rehabilitation device includes at least one cervical vertebrae cell, a head cell connected to the at least one cervical vertebrae cell, an electric inflation portion and a control portion. The method includes a preparing step, a setting inflation processes step, an inflating step, and a completion step. One or more cervical vertebrae cells are inflatable and positioned between a support position and an operation position to support and rehabilitate the user's neck. The head cell is provided to support the user's head. So, the cervical vertebrae cells can support the user's head and neck with rehabilitation functions. The head cell is an auxiliary for support the user's head. It is easy to carry. This invention could be remote controlled. It contains the far infra-red heating function. In addition, there is a pose balance feature.

### FIELD OF THE INVENTION

The present invention relates to an inflation type cervical vertebrae rehabilitation device and a method for using the device.

[1] The cervical vertebrae cells can support the user's head and neck with rehabilitation functions.

[2] The head cell is an auxiliary for support the user's head.

[3] It is easy to carry.

[4] This invention could be remote controlled.

[5] It contains the far infra-red heating function.

[6] There is a pose balance feature.

### BACKGROUND OF THE INVENTION

A conventional s used to protect the cervical vertebrae and which includes a steel frame coated with soft pad. However, the conventional cervical collar makes the user to feel uncomfortable. A portable cervical vertebrae traction device is developed and shown in FIG. 16 and generally includes a frame 92 which is fixed to a wall and a cable 93 goes around the frame 92 and one end of the cable 93 is connected with a weight 94 and the other end of the cable 93 is connected with a strap 95. The user 91 puts his/her chin 913 on the strap 95 so that the cervical vertebrae can be pulled by the weight.

Nevertheless, the conventional one has many drawbacks listed as follows. The portable cervical vertebrae traction device cannot be used for the user who wears the conventional cervical collar. The treatment cannot be done by a periodically and automatically inflation member. In addition, there are no inflation members to support the cervical vertebrae and the device cannot be controlled by a wireless device.

The present invention intends to provide an inflation type cervical vertebrae rehabilitation device and a method for using the device to improve the shortcomings of the above-mentioned device and method.

### SUMMARY OF THE INVENTION

The object of the present invention is to provide an inflation type cervical vertebrae rehabilitation device and a method for using the device. In which, the cervical vertebrae cells can support the user's head and neck with rehabilitation functions. The head cell is an auxiliary for support the user's head. It is easy to carry. This invention could be remote controlled. It contains the far infra-red heating function. In addition, there is a pose balance feature.

In order to achieve the above-mentioned objects, the present invention is provided. The present invention relates to an inflation type cervical vertebrae rehabilitation device and comprises at least one cervical vertebrae cell for mounting a neck of a user and having a contact area for being in contact with the neck. The at least one cervical vertebrae cell is positioned between at least one support position and at least one operation position. A head cell has a contact surface and is connected to the at least one cervical vertebrae cell. The head cell is positioned between an auxiliary support position and an auxiliary adjusting position. The contact surface supports the user's head and adjusts height of the head when the user lies down. An electric inflation portion inflates or deflates the at least one cervical vertebrae cell and the head cell. A control portion controls the electric inflation portion.

The method for using the inflation type cervical vertebrae rehabilitation device comprises a preparing step; a setting inflation processes step; an inflating step; and a completion step.

The present invention further comprises a heat unit, an audio unit, at least one orientation detection unit and a wireless unit so as to provide warming feature, audio instruction feature, pose balance feature and remote-controlled feature.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1A shows the inflation type cervical vertebrae rehabilitation device and the wireless remote control portion of the present invention

FIG. 1B shows the inflation type cervical vertebrae rehabilitation device and the control portion with wire of the present invention

FIG. 2 shows that the cervical vertebrae cells deflated;

FIG. 3 shows that the cervical vertebrae cells are mounted to the user's neck;

FIG. 4 shows cervical vertebrae cells each have a heat unit;

FIG. 5 shows the first way of the use of the inflation type cervical vertebrae rehabilitation device of the present invention;

FIG. 6 shows the second way of the use of the inflation type cervical vertebrae rehabilitation device of the present invention;

FIG. 7 shows the user's pose is not tilt when wearing the inflation type cervical vertebrae rehabilitation device of the present invention;

FIG. 8 shows the user's pose is tilted when wearing the inflation type cervical vertebrae rehabilitation device of the present invention;

FIG. 9 shows that the orientation detection unit on the cervical vertebrae cells and the user's body tilts an angle;

FIG. 10 shows the block diagram of the inflation type cervical vertebrae rehabilitation device of the present invention;

FIG. 11 shows the operation processes of the first action of the inflation type cervical vertebrae rehabilitation device of the present invention;

FIG. 12 shows the operation processes of the second action of the inflation type cervical vertebrae rehabilitation device of the present invention;

FIG. 13 shows the operation processes of the third action of the inflation type cervical vertebrae rehabilitation device of the present invention;

FIG. 14 shows the operation processes of the fourth action of the inflation type cervical vertebrae rehabilitation device of the present invention;

FIG. 15 shows the flow chart of the method of the present invention, and

FIG. 16 shows the conventional portable cervical vertebrae traction device.

### DETAILED DESCRIPTION OF THE PREFERRED EMBODIMENT

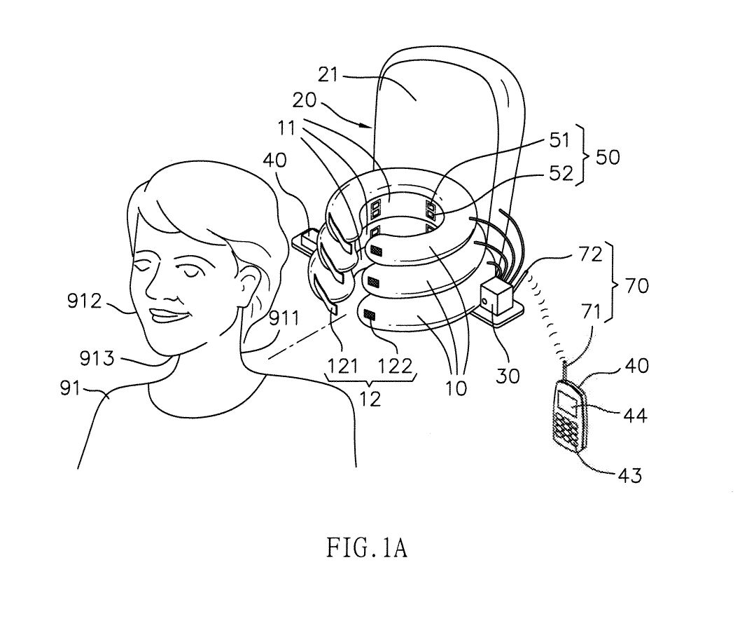

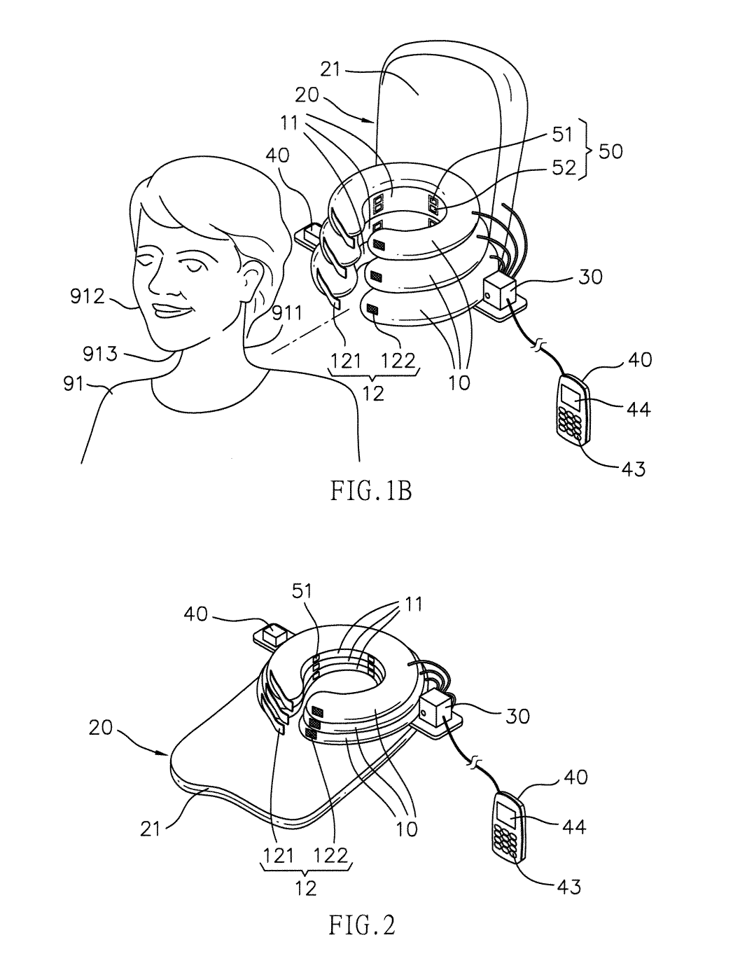

Referring to FIGS. 1A, 2, 3 and 5, the inflation type cervical vertebrae rehabilitation device of the present invention comprises at least one cervical vertebrae cell 10 for mounting the neck 911 of a user 91, and the at least one cervical vertebrae has a contact area 11 which is in contact with the neck 911. The at least one cervical vertebrae cell 10 is positioned between at least one support position P1 and at least one operation position P2 to support and rehabilitate the user' s cervical vertebrae.

A head cell 20 has a contact surface 21 and is connected to the at least one cervical vertebrae cell 10. The head cell 20 is positioned between an auxiliary support position P3 and an auxiliary adjusting position P4. The contact surface 21 supports the head 912 of the user 91 and adjusts height of the head 912 of the user 91 when the user 91 lies down as shown in FIG. 6.

An electric inflation portion 30 is used to inflate (FIG. 1A) or deflate (FIG. 2) the at least one cervical vertebrae cell 10 and the head cell 20.

A control portion 40 controls the electric inflation portion 30 to proceed the actions of inflating or deflating the at least one cervical vertebrae cell 10 and the head cell 20.

The at least one cervical vertebrae cell 10 includes three cells which are C-shaped cells and each have a positioning member 12 located at en opening thereof. The positioning member 12 is a loop-and-hook member including a first part 121 and a second part 122, so that the cervical vertebrae cells 10 are secured on the neck 911 of the user 91.

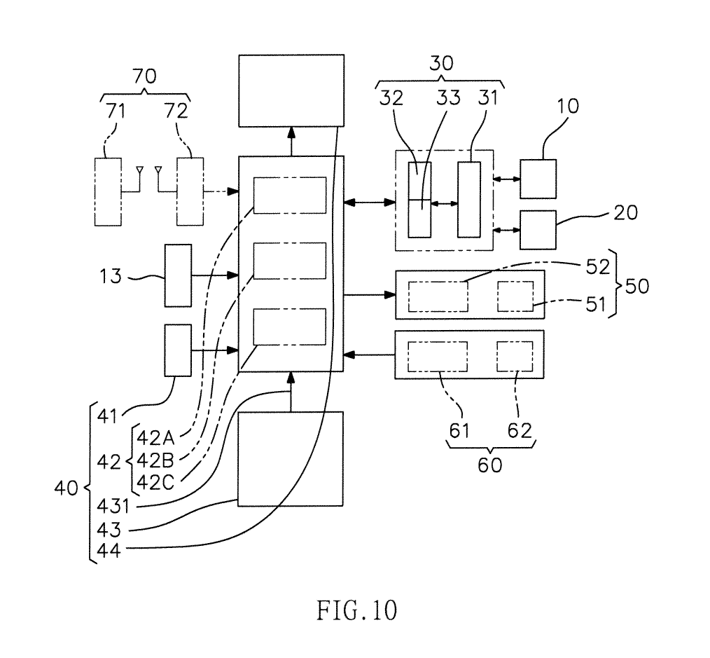

As shown in FIG. 10, the electric inflation portion 30 comprises an inflation unit 31 which is a motor so as to inflate or deflate the at least one cervical vertebrae cell 10 and the head cell 20. A motor control module 32 controls the motor of the inflation unit 31 and a pressure detection module 33 detects pressure in the at least at least one cervical vertebrae cell 10 and the head cell 20. The result of the detection is transferred to the control portion 40 which controls the at least one cervical vertebrae cell 10 and the head cell 20 and the head cell 20 to be inflated or deflated.

The control portion 40 comprises a power unit 41 which includes batteries providing power to the control portion 40. A control circuit 42 has at least one processing unit 42A, a random access memory (RAM) 42B, and a read only memory (ROM) 42C. The control circuit 42 controls the control portion 40 to control the inflation portion 30. An input unit 43 inputs at least one control signal 431 to the control circuit 42 and is a human-machine interface. A display unit 44 displays the control signal 431 from the input unit 43.

The at least one cervical vertebrae cell 10 has at least one orientation detection unit 13 connected thereto which is a mercury switch (FIG. 9) and detects an tilt angle of the user 91 if the angle reaches the critical angle and submits a warning signal to the control portion 40.

A heat unit 50 includes multiple heating members 51 which are far infra-red members. A temperature detection module 52 detects temperature transferred to the at least one cervical vertebrae cell 10 from the multiple heating members 51 and submits a signal to the control portion 40 to control the multiple heating members 51. An audio unit 60 includes an audio control module 61 and a speaker 62 so as to generate audio signals. A wireless unit 70 includes at least one wireless submitting member 71 and a wireless receiving member 72.

The present invention may perform the following modes:

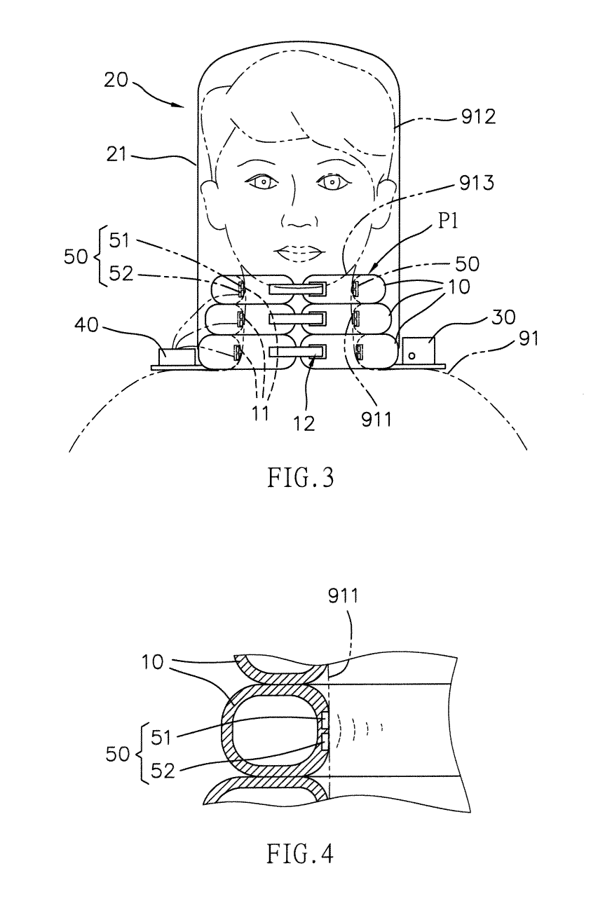

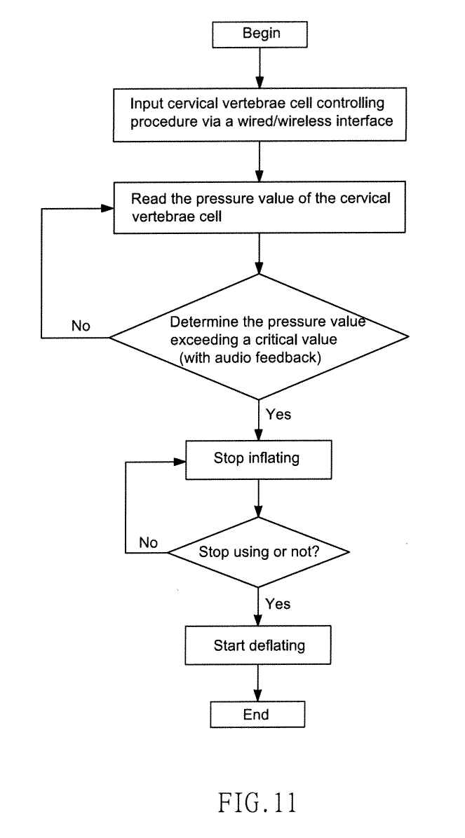

1. Cervical vertebrae support mode. As shown in FIGS. 3 and 11, the cervical vertebrae cells 10 are mounted to the user's neck 911 and the control processes are input via the input unit 43 of the control portion 40 by way of wireless way as shown in FIG. 1A or wired way as shown in FIG. 1B. The electrical inflation portion 30 inflates the cervical vertebrae cells 10 by preset pressure and the pressure detection module 33 detects pressure in the cervical vertebrae cells 10 to judge the pressure reaches the critical value or not (may be cooperated with the audio unit 60). If the value reaches the critical value, then the inflation action is stopped. The contact areas 11 support the user's neck 911 when in the support position P1. The cervical vertebrae cells 10 are deflated when not in use.

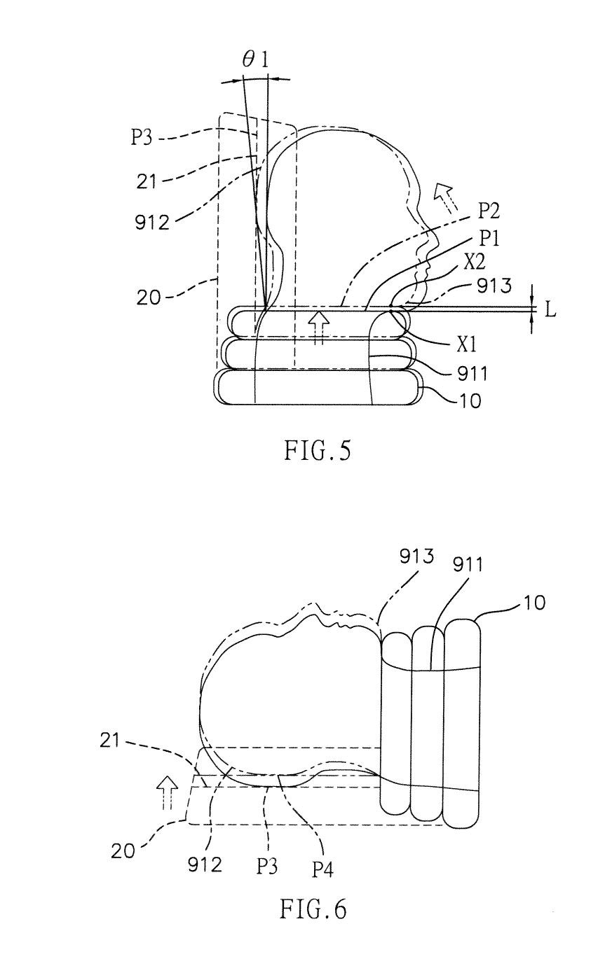

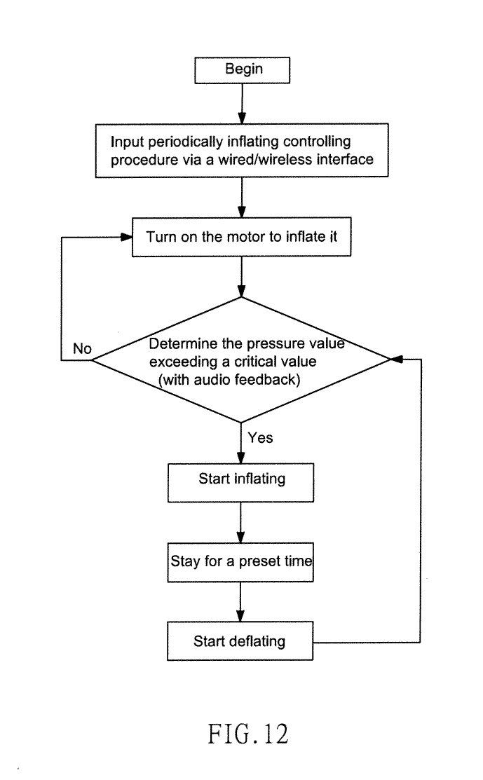

2. Cervical vertebrae rehabilitation mode. As shown in FIGS. 5 and 12, the cervical vertebrae cells 10 are mounted to the user's neck 911 and the periodically inflating processes are input via the input unit 43 of the control portion 40, the inflation portion 30 is then activated to inflate the cervical vertebrae cells 10. The pressure detection module 33 detects pressure in the cervical vertebrae cells 10 to judge the pressure reaches the critical value or not (may be cooperated with the audio unit 60). If the value reaches the critical value, then the inflation action is stopped. The cervical vertebrae cells 10 are deflated after a pre-set period of time, and then to inflate again. By the repeated inflating and deflating actions, the cervical vertebrae cells 10 are positioned between the support position P1 and operation position P2. The user's chin 913 is moved between the relax point X1 and push point X2, wherein a height “L” is defined between the points X1 and X2. Accordingly, the user's body swings an angle θ while the head 912 is stationary.

3. Pillow adjustment mode. As shown in FIG. 6, the cervical vertebrae cells 10 are mounted to the user's neck 911 and the control processes are input via the input unit 43 of the control portion 40, the electrical inflation portion 30 inflates the head cells 20 to assist the position change between the auxiliary support position P3 and auxiliary adjusting position P4. The user's head 912 is comfortably supported and the neck 911 is adjusted to be well supported to obtain a good sleep.

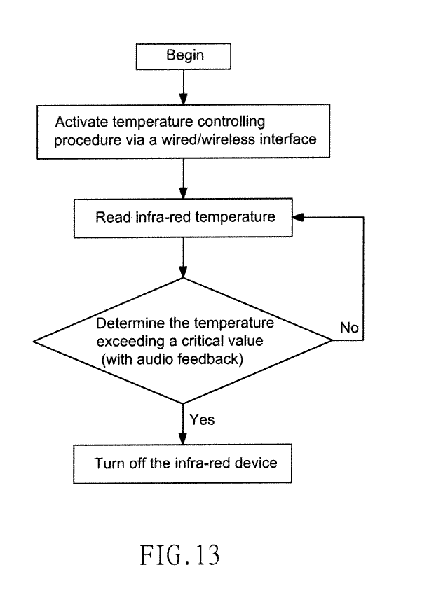

4. Far infra-red heating mode. As shown in FIGS. 3, 4 and 13, the cervical vertebrae cells 10 are mounted to the user's neck 911 and the control processes are input via the input unit 43 of the control portion 40. The heating members 51 of the heat portion 50 are activated to generate proper heat to the user's neck 911 to have physical treatment to the user's neck.

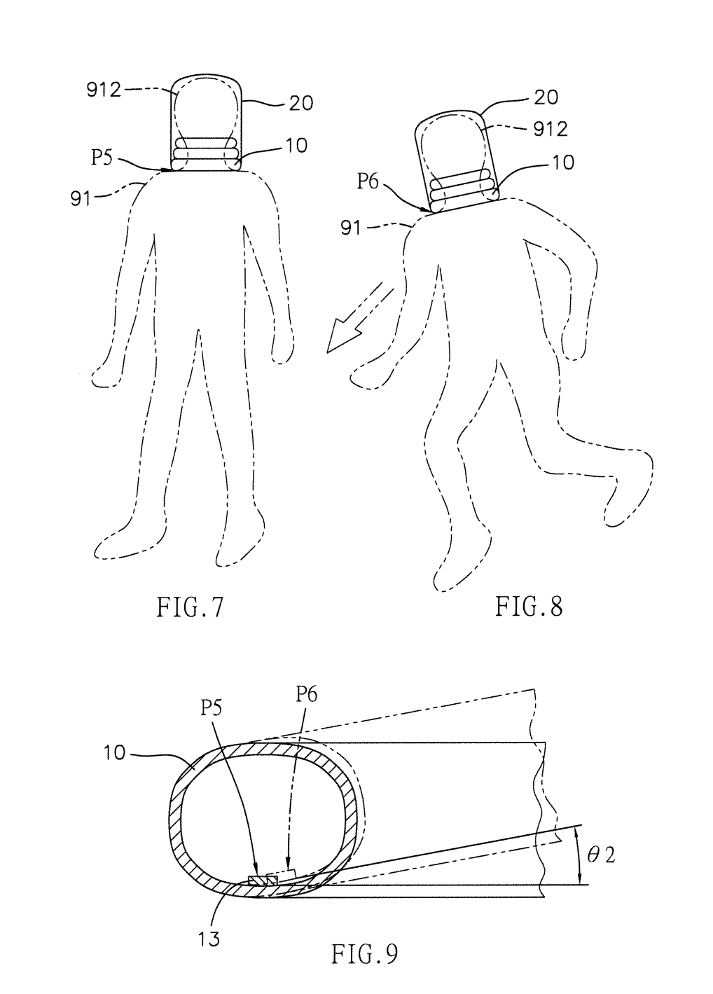

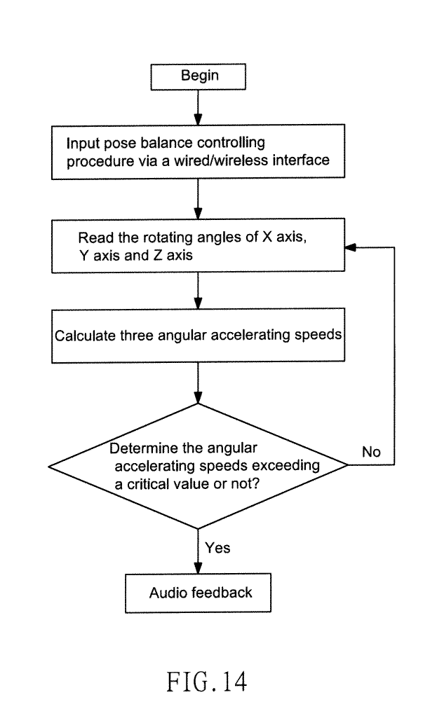

5. Pose balance mode. As shown in FIGS. 7 and 14, when the pose control processes are input via the input unit 43 of the control portion 40. If the user suddenly changes his/her pose as shown in FIG. 8 wherein the head 912 tilts too much, the orientation detection unit 13 is suddenly changed its position from the balance position P5 to a tilt position P6 as shown in FIG. 9, the orientation detection unit 13 tilts an angle θ2 and generates a rotational angle in X axis (or Y axis or Z axis). The change is sent to the control portion 40 to be calculated, it the change is so significant and over the critical value, an audio warning message is generated to remind the user 91 to adjust the pose to prevent falling.

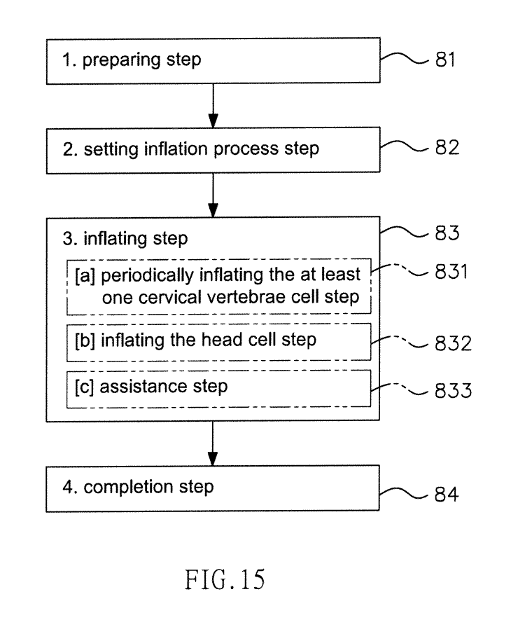

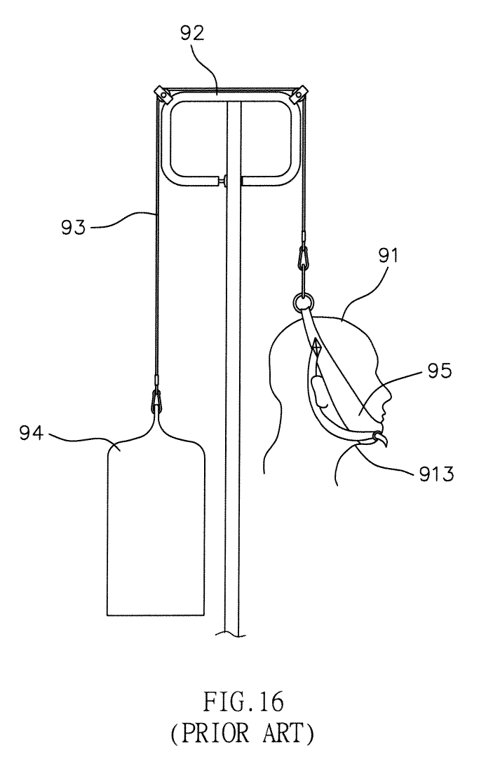

FIG. 15 shows the method for using the inflation type cervical vertebrae rehabilitation device, and comprises the following steps:

A preparing step 81: preparing at least one cervical vertebrae cell 10, a head cell 20 connected to the at least one cervical vertebrae cell 10, an electric inflation portion 30 and a control portion 40, the at least one cervical vertebrae having a contact area 11 in contact with the neck 911, the head cell 20 having a contact surface 21 and connected to the at least one cervical vertebrae cell 10, the electric inflation portion 30 being controlled by the control portion 40 so as to inflate or deflate the at least one cervical vertebrae cell 10 and the head cell 20;

A setting inflation processes step 82: the control portion 40 having an input unit 43 to selectively demand the electric inflation portion 30 to inflate at least one of at least one cervical vertebrae cell 10 and the head cell 20;

An inflating step 83:

(a) A periodically inflating cervical vertebrae cell step 831: the electric inflation portion 30 inflating the at least one cervical vertebrae cell 10 by the input unit 43 of the control portion 40 so that the at least one cervical vertebrae cell 10 is positioned between at least one support position P1 and at least one operation position P2. The user's chin 913 is moved between the relax point X1 and push point X2, wherein a height “L” is defined between the points X1 and X2 as shown in FIG. 5, such that the user's body swings an angle 0 1 while the head 912 is stationary;

(b) An inflating the head cell step 832: the electric inflation portion 30 inflating or deflating the head cell 20 by the input unit 43 of the control portion 40 so that the head cell 20 is positioned between at least one auxiliary support position P3 and at least one auxiliary adjusting position P4, the contact surface 21 adapted to support the head 912 of the user 91 and adjust height of the head 912 of the user 91 when the user lies down as shown in FIG. 6, and

A completion step 84: removing the at least one cervical vertebrae cell 10 from the user 91.

During the inflating step 83, the following modes are obtained:

(c) An assistance step 833 and the assistance step 833 may have the three modes:

Cervical vertebrae support mode:

When the cervical vertebrae cells 10 are inflated by the pre-set pressure by the electric inflation portion 30, and the pressure detection module 33 detects the pressure in the cervical vertebrae cells 10 to judge the pressure reaches the critical value or not (may be cooperated with the audio unit 60). If the value reaches the critical value, then the inflation action is stopped. The contact areas 11 support the user's neck 911 when in the support position P1.

Far infra-red mode:

As shown in FIGS. 3, 4 and 13, the cervical vertebrae cells 10 are inflated so that the contact areas 11 contact the user's neck 911 and the control processes are input via the input unit 43 of the control portion 40 so that the heating members 51 of the heat portion 50 are activated to generate proper heat to the user's neck 911 to have physical treatment to the user's neck.

Pose balance mode:

As shown in FIGS. 7 and 14, when the pose control processes are input via the input unit 43 of the control portion 40. If the user suddenly changes his/her pose as shown in FIG. 8 wherein the head 912 tilts too much, the orientation detection unit 13 is suddenly changed its position from the balance position P5 to a tilt position P6 as shown in FIG. 9, the orientation detection unit 13 tilts an angle θ2 and generates a rotational angle in X axis (or Y axis or Z axis). The change is sent to the control portion 40 to be calculated, it the change is so significant and over the critical value, an audio warning message is generated to remind the user 91 to adjust the pose to prevent falling.

The advantages and functions of this invention can be summarized as follows.

[1] The cervical vertebrae cells can support the user's head and neck with rehabilitation functions.

[2] The head cell is an auxiliary for support the user's head.

[3] It is easy to carry.

[4] This invention could be remote controlled.

[5] It contains the far infra-red heating function.

[6] There is a pose balance feature.

While we have shown and described the embodiment in accordance with the present invention, it should be clear to those skilled in the art that further embodiments may be made without departing from the scope of the present invention.

What is claimed is:

1. An inflation type cervical vertebrae rehabilitation device comprising:
at least one cervical vertebrae cell for mounting a neck of a user, the at least one cervical vertebrae having a contact area adapted to be in contact with the neck, the at least one cervical vertebrae cell being positioned between at least one support position and at least one operation position;
a head cell having a contact surface and connected to the at least one cervical vertebrae cell, the head cell being positioned between an auxiliary support position and an auxiliary adjusting position, the contact surface adapted to support a head of the user and adjust height of the head of the user when the user lies down;
an electric inflation portion for inflating or deflating the at least one cervical vertebrae cell and the head cell, and
a control portion for controlling the electric inflation portion to inflate or deflate.

2. The device as claimed in claim 1, wherein the at least one cervical vertebrae cell includes three cells which are C-shaped cells and each have a positioning member located at en opening thereof, the positioning member is a loop-and-hook member so that the cervical vertebrae cells are adapted to be secured on the neck of the user.

3. The device as claimed in claim 1, wherein the electric inflation portion comprises an inflation unit which is a motor so as to inflate or deflate the at least one cervical vertebrae cell and the head cell, a motor control module controls the motor of the inflation unit, a pressure detection module detects pressure in the at least at least one cervical vertebrae cell and the head cell and then sends to the control portion.

4. The device as claimed in claim 1, wherein the control portion comprises a power unit which includes batteries providing power to the control portion, a control circuit has at least one processing unit, a random access memory, and a read only memory, the control circuit controls the control portion to control the inflation portion, an input unit inputs at least one control signal to the control circuit and is a human-machine interface, and a display unit displays the control signal from the input unit.

5. The device as claimed in claim 1, wherein the at least one cervical vertebrae cell has at least one orientation detection unit connected thereto which is a mercury switch and detects an tilt angle of the user and submits a warning signal to the control portion, a heat unit includes multiple heating members which are far infra-red members, a temperature detection module detects temperature transferred to the at least one cervical vertebrae cell from the multiple heating members and submits a signal to the control portion to control the multiple heating members, an audio unit includes an audio control module and a speaker so as to generate audio signals, a wireless unit includes at least one wireless submitting member and a wireless receiving member.

6. A method for using the inflation type cervical vertebrae rehabilitation device, comprising:
a preparing step: preparing at least one cervical vertebrae cell, a head cell connected to the at least one cervical vertebrae cell, an electric inflation portion and a control portion, the at least one cervical vertebrae having a contact area adapted to be in contact with the neck, the head cell having a contact surface and connected to the at least one cervical vertebrae cell, the electric inflation portion being controlled by the control portion so as to inflate or deflate the at least one cervical vertebrae cell and the head cell;
a setting inflation processes step: the control portion having an input unit to selectively control the electric inflation portion to inflate at least one of at least one cervical vertebrae cell and the head cell;
an inflating step comprising:
(a) periodically inflating the at least one cervical vertebrae cell: the electric inflation portion inflating the at least one cervical vertebrae cell by the input unit of the control portion so that the at least one cervical vertebrae cell is positioned between at least one support position and at least one operation position such that the user's body swings an angle while the head is stationary;
(b) inflating the head cell: the electric inflation portion inflating or deflating the head cell by the input unit of the control portion so that the head cell is positioned between at least one auxiliary support position and at least one auxiliary adjusting position, the contact surface adapted to support the head of the user and adjust height of the head of the user when the user lies down; and
a completion step: removing the at least one cervical vertebrae cell from the user.

7. The method as claimed in claim 6, wherein the at least one cervical vertebrae cell includes three cells which are C-shaped cells and each have a positioning member located at en opening thereof, the positioning member is a loop-and-hook member so that the cervical vertebrae cells are adapted to be secured on the neck of the user, the electric inflation portion comprises an inflation unit which is a motor so as to inflate or deflate the at least one cervical vertebrae cell and the head cell, a motor control module controls the motor of the inflation unit, a pressure detection module detects pressure in the at least at least one cervical vertebrae cell and the head cell and the head cell and then sends to the control portion, the control portion comprises a power unit which includes batteries providing power to the control portion, a control circuit has at least one processing unit, a random access memory, and a read only memory, the control circuit controls the control portion to control the inflation portion, an input unit inputs at least one control signal to the control circuit and is a human-machine interface, a display unit displays the control signal from the input unit.

8. The method as claimed in claim 6, wherein the at least one cervical vertebrae cell has at least one orientation detection unit connected thereto which is a mercury switch and detects an tilt angle of the user and submits a warning signal to the control portion, a heat unit includes multiple heating members which are far infra-red members, a temperature detection module detects temperature transferred to the at least one cervical vertebrae cell from the multiple heating members and submits a signal to the control portion to control the multiple heating members, an audio unit includes an audio control module and a speaker so as to generate audio signals, a wireless unit includes at least one wireless submitting member and a wireless receiving member.
